(19) United States
(12) Patent Application Publication
(10) Pub. No.: US 2026/0007860 A1
(43) Pub. Date: Jan. 8, 2026

(54) SECUREMENT DEVICE FOR CATHETER, KIT, AND METHOD OF USE

(71) Applicant: Solventum Intellectual Properties Company, Maplewood, MN (US)

(72) Inventors: Ganesh P.D. Kannan, Bangalore (IN); Alan R. Dombrowski, Woodbury, MN (US); Krystal J. Scheibel, Minneapolis, MN (US); James M. Sieracki, Plymouth, MN (US); Timothy J. Nies, Stillwater, MN (US)

(73) Assignee: Solventum Intellectual Properties Company, Maplewood, MN (US)

(21) Appl. No.: 19/127,187

(22) PCT Filed: Oct. 30, 2023
(86) PCT No.: PCT/IB2023/060924

§ 371 Date: May 5, 2025

Related U.S. Application Data
(60) Provisional application No. 63/423,413, filed on Nov. 7, 2022.

(51) Int. Cl. A61M 25/02

(52) U.S. Cl. CPC A61M 25/02; A61M 2025/0253

(57) ABSTRACT

A securement device includes a base; a base mount fixedly attached to the base and including a coupling member and a retaining member spaced apart from the coupling member; and a swing arm including an elongate member pivotally coupled to the coupling member and a cannula securement member connected to the elongate member and spaced apart from the coupling member. The cannula securement member is configured to removably secure the cannula thereto and to be detachably coupled to the retaining member. The securement device includes a maintenance state in which the cannula securement member is detached from the retaining member and the swing arm is pivoted away from the retaining member, such that the cannula securement member is spaced apart from the retaining member while being secured to the cannula.

### TECHNICAL FIELD

The present disclosure relates to a securement device for a catheter, a kit, and a method of using the securement device with the catheter.

### BACKGROUND

Catheters may be used for various purposes, such as feeding, air supply, and/or liquid removal. Catheters typically include a cannula that is inserted into a skin of a patient at an insertion site. In some cases, the catheters may be used to administer medications and fluids to the patient. In many instances, the catheters may need to be secured to the skin of the patient to prevent movement of the catheter relative to the skin. For example, the catheters may need to be secured to the skin of the patient to prevent the catheters from being pulled out, or otherwise moved in ways that may adversely affect functioning of the catheters.

The catheters are generally secured to the skin of the patient using conventional securement devices, such as tapes, patches, and sutures. However, the conventional securement devices may not properly secure the catheters to the skin. Specifically, the conventional securement devices may not properly stabilize the catheters, thereby allowing undesirable movement of the catheters during use. Therefore, in some cases, use of the conventional securement devices may result in various complications, such as dislodgment and accidental removal of the catheters from the skin of the patient, phlebitis, extravasation/infiltration, leakage, occlusion, and bloodstream infections. Moreover, the conventional securement devices may need to be removed from the skin in order to carry out a maintenance procedure of the skin proximal to the insertion site, which may be inconvenient to the patient. Removing the conventional securement devices from the skin may further cause “pistoning” of the cannula, which may cause discomfort, and in some cases, result in injuries.

### SUMMARY

In a first aspect, the present disclosure provides a securement device for a catheter having a cannula configured to be inserted into a skin of a patient at an insertion site. The securement device includes a base configured to be detachably secured to the skin of the patient. The securement device further includes a base mount fixedly attached to the base. The base mount includes a coupling member and a retaining member spaced apart from the coupling member. The securement device further includes a swing arm including an elongate member pivotally coupled to the coupling member of the base mount. The swing arm further includes a cannula securement member connected to the elongate member and spaced apart from the coupling member. The cannula securement member is configured to removably secure the cannula thereto. The cannula securement member is further configured to be detachably coupled to the retaining member. Upon detachment of the cannula securement member from the retaining member, the swing arm is pivotable relative to the base mount. In a secured state of the securement device, the cannula is removably secured to the cannula securement member and the cannula securement member is detachably coupled to the retaining member. In a maintenance state of the securement device, the cannula securement member is detached from the retaining member and the swing arm is pivoted away from the retaining member, such that the cannula securement member is spaced apart from the retaining member while being secured to the cannula.

In a second aspect, the present disclosure provides a kit. The kit includes a catheter including a cannula configured to be inserted into a skin of a patient at an insertion site. The kit further includes a securement device. The securement device includes a base configured to be detachably secured to the skin of the patient. The securement device further includes a base mount fixedly attached to the base. The base mount includes a coupling member and a retaining member spaced apart from the coupling member. The securement device further includes a swing arm including an elongate member pivotally coupled to the coupling member of the base mount. The swing arm further includes a cannula securement member connected to the elongate member and spaced apart from the coupling member. The cannula securement member is configured to removably secure the cannula thereto. The cannula securement member is further configured to be detachably coupled to the retaining member. Upon detachment of the cannula securement member from the retaining member, the swing arm is pivotable relative to the base mount. In a secured state of the securement device, the cannula is removably secured to the cannula securement member and the cannula securement member is detachably coupled to the retaining member. In a maintenance state of the securement device, the cannula securement member is detached from the retaining member and the swing arm is pivoted away from the retaining member, such that the cannula securement member is spaced apart from the retaining member while being secured to the cannula.

In a third aspect, the present disclosure provides a method of using a securement device with a catheter having a cannula inserted into a skin of a patient at an insertion site. The securement device includes a base configured to be detachably secured to the skin of the patient, a base mount fixedly attached to the base and having a coupling member and a retaining member spaced apart from the coupling member, and a swing arm having an elongate member pivotally coupled to the coupling member of the base mount and a cannula securement member connected to the elongate member and spaced apart from the coupling member. The method includes removably securing the base to the skin of the patient. The method further includes removably securing the cannula of the catheter to the cannula securement member. The method further includes detachably securing the cannula securement member to the retaining member. The method further includes detaching the cannula securement member from the retaining member, such that the swing arm is pivotable relative to the retaining member. The method further includes pivoting the swing arm away from the retaining member, such that the cannula securement member is spaced apart from the retaining member while being secured to the cannula.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic perspective view of a catheter according to an embodiment of the present disclosure;

FIG. 2 is a schematic top perspective view of a securement device according to an embodiment of the present disclosure;

FIG. 3 is a schematic exploded perspective view of the securement device according to an embodiment of the present disclosure;

FIG. 4 is a schematic sectional view of a portion of the securement device taken along a line 1-1 of FIG. 2 according to an embodiment of the present disclosure;

FIGS. 5A-5E are schematic perspective views of the securement device depicting a method of using the securement device according to an embodiment of the present disclosure;

FIG. 6 is a schematic block diagram of a kit according to an embodiment of the present disclosure; and

FIG. 7 is a flowchart depicting various steps of a method of using the securement device with the catheter according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B.”

Unless specified or limited otherwise, the terms “attached,” “connected,” and variations thereof, are used broadly and encompass both direct and indirect attachments, connections, and couplings.

As used herein, the terms “layer,” “sheet,” and “dressing,” or variations thereof, are used to describe an article having a thickness that is small relative to its length and width.

As used herein, the term “polymer” refers to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The term “copolymer” refers to a polymeric material prepared from at least two different monomers.

As used herein, the term “inserted into skin” when used in conjunction with the terms “cannula” or “catheter” refers to insertion of a catheter or a cannula through the skin of a patient. For example, a distal end of a catheter or a cannula may be located at any desired anatomical site including, as non-limiting examples, within the lumens of veins and/or arteries, within the atria and the ventricles of the heart, within the ventral body cavity, within the spinal canal, within the subarachnoid space, within the intracranial space, and within fat and/or muscle tissues.

As used herein, the term “insertion site” refers to a location at which a cannula of a catheter is inserted into a skin of a patient.

As used herein, the term “maintenance procedure” refers generally to a procedure that includes cleaning and/or sanitizing of a skin of a patient proximal to an insertion site. The maintenance procedure may be carried out by a medical practitioner at regular intervals during, for example, an intravenous therapy.

As used herein, the term “pistoning” or “pistoning movement” refers to a movement of a cannula of a catheter relative to an insertion site.

The present disclosure provides a securement device for a catheter having a cannula configured to be inserted into a skin of a patient at an insertion site. The securement device includes a base configured to be detachably secured to the skin of the patient. The securement device further includes a base mount fixedly attached to the base. The base mount includes a coupling member and a retaining member spaced apart from the coupling member. The securement device further includes a swing arm including an elongate member pivotally coupled to the coupling member of the base mount. The swing arm further includes a cannula securement member connected to the elongate member and spaced apart from the coupling member. The cannula securement member is configured to removably secure the cannula thereto. The cannula securement member is further configured to be detachably coupled to the retaining member. Upon detachment of the cannula securement member from the retaining member, the swing arm is pivotable relative to the base mount. In a secured state of the securement device, the cannula is removably secured to the cannula securement member and the cannula securement member is detachably coupled to the retaining member. In a maintenance state of the securement device, the cannula securement member is detached from the retaining member and the swing arm is pivoted away from the retaining member, such that the cannula securement member is spaced apart from the retaining member while being secured to the cannula.

In the secured state, the securement device of the present disclosure may firmly secure the catheter to the skin of the patient. The securement device may therefore prevent various complications, such as phlebitis, extravasation/infiltration, leakage, occlusion, and bloodstream infections that may otherwise occur due to movement of the catheter relative to the skin.

In the maintenance state, the securement device of the present disclosure may provide a medical practitioner with access to the insertion site. The medical practitioner may perform a maintenance procedure at the insertion site in the maintenance state of the securement device. The securement device may allow the medical practitioner to carry out the maintenance procedure without needing to remove or detach the securement device from the skin.

The cannula securement member of the securement device may be detached from the retaining member, and the swing arm may be pivoted away from the retaining member to conveniently bring the securement device into the maintenance state from the secured state. The securement device may therefore facilitate performing the maintenance procedure at the insertion site. Moreover, the securement device may transition between the secured state and the maintenance state without causing a pistoning movement of the cannula.

In some examples, the base of the securement device may be detachably secured to the skin of the patient via a stretch-release adhesive. The stretch-release adhesive may facilitate removal of the base from the skin of the patient. In other words, the base may be easily removed from the skin of the patient without causing discomfort to the patient.

Therefore, the securement device may firmly secure the catheter to the skin of the patient in the secured state, thereby inhibiting undesirable movement of the catheter relative to the skin, may facilitate the maintenance procedure at the insertion site in the maintenance state, and may facilitate removal of the securement device after use.

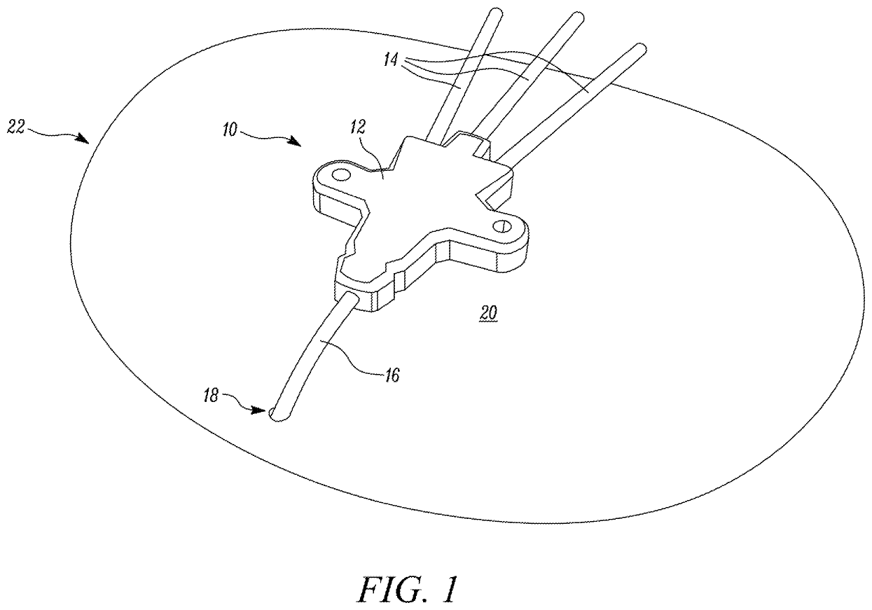

Referring now to the Figures. FIG. 1 illustrates a schematic perspective view of a catheter 10 according to an embodiment of the present disclosure. In FIG. 1, the catheter 10 is partially disposed on a skin 20 of a patient 22. In some cases, the patient 22 may be undergoing an intravenous therapy.

The catheter 10 may include a hub 12, one or more lumens 14, and a cannula 16. The one or more lumens 14 may be at least partially received within the hub 12. The one or more lumens 14 are partially shown in FIG. 1. In the illustrated embodiment of FIG. 1, the one or lumens 14 include three lumens 14. In other words, in the illustrated embodiment of FIG. 1, the catheter 10 is a triple lumen catheter. However, a number of the one or more lumens 14 may vary based on desired application and/or infusion attributes. The one or more lumens 14 may be fluidly connected with the cannula 16. As shown in FIG. 1, the cannula 16 may be at least partially received within the hub 12. Further, the cannula 16 may be inserted into the skin 20 of the patient 22 at an insertion site 18.

In some embodiments, the catheter 10 may include a central venous catheter (CVC). The CVC may be referred to as a central line, a central venous line, or a central venous access catheter. The CVC may be used to access large, centrally located veins, which is often required for critically ill patients, for patients requiring prolonged intravenous therapies for reliable vascular access, and to administer fluids that may harm smaller peripheral veins. Therefore, at least a portion of the cannula 16 may be inserted into veins located at a neck (e.g., an internal jugular vein), into veins located at a chest (e.g., a subclavian vein or axillary vein), and into veins located at a groin (e.g., a femoral vein) of the patient 22. In some embodiments, the catheter 10 may include a peripherally inserted central catheter (PICC). The PICC may be suitable for insertion into veins located at an arm of the patient 22. It may be noted that the catheter 10 may include any suitable type of catheter, which may be selected based on desired application attributes.

The catheter 10 may be used to administer an intravenous therapy (e.g., medication or fluids for parenteral nutrition), to obtain blood for analysis, and to provide an access point for blood-based treatments. such as dialysis or apheresis. The catheter 10 may further be used to obtain blood tests (e.g., a “central venous oxygen saturation”), administer fluid or blood products for a large volume resuscitation, and measure a central venous pressure.

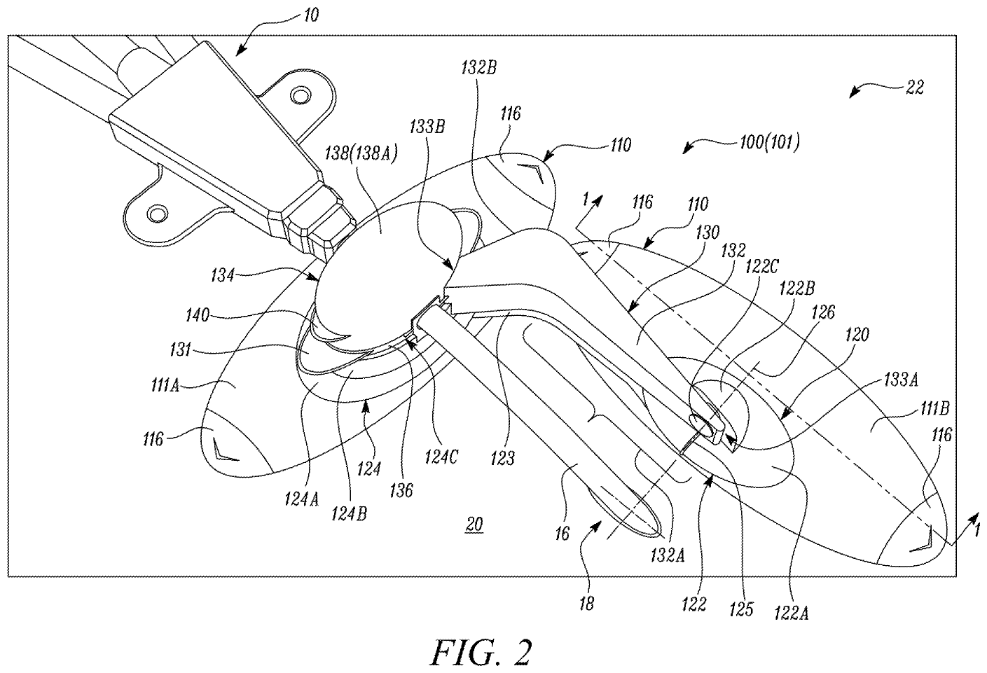

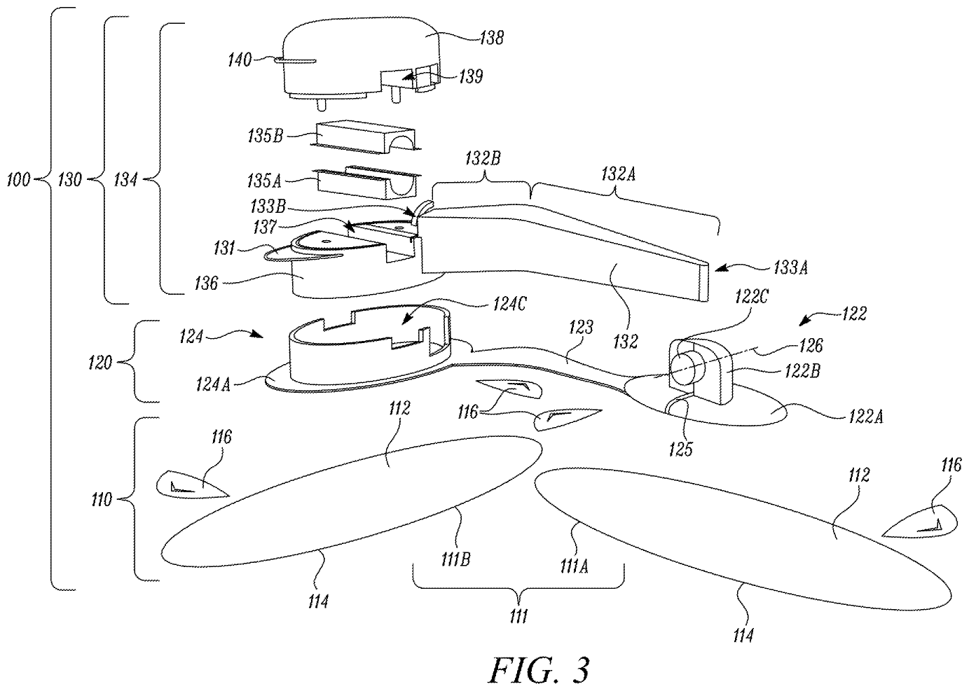

FIG. 2 illustrates a schematic top perspective view of a securement device 100 for the catheter 10 according to an embodiment of the present disclosure. FIG. 3 illustrates a schematic exploded perspective view of the securement device 100 according to an embodiment of the present disclosure.

In FIG. 2, the cannula 16 is inserted into the skin 20 of the patient 22 at the insertion site 18. Furthermore, the securement device 100 is in a secured state 101. As will be further discussed below, the securement device 100 may secure the catheter 10 to the skin 20 in the secured state 101.

Referring to FIGS. 2 and 3, the securement device 100 includes a base 110 configured to be detachably secured to the skin 20 of the patient 22. The base 110 may be selectively secured to and detached from the skin 20 as desired. The base 110 may be detachably secured to the skin 20 by any suitable method, such as, for example, adhesives, tapes, hook-and-loop fasteners, and the like.

As shown in FIG. 3, the base 110 may include a substrate 111 including a first major surface 112, and a second major surface 114 opposite to the first major surface 112. The second major surface 114 may be configured to face the skin 20.

The substrate 111 may be made of a stretchable material that can conform to undulated surfaces (e.g., the skin 20 of the patient 22). The substrate 111 may be configured to exhibit high elasticity. In some embodiments, a modulus of elasticity of the substrate 111 may be at least about 2,500 pound-force per square inch (psi) or at least about 3,000 psi. In some embodiments, the modulus of elasticity of the substrate 111 may be less than about 50,000 psi. In some embodiments, the modulus of elasticity of the substrate 111 may be between about 5,000 psi and about 30,000 psi. In some embodiments, the substrate 111 may have a high tensile strength, a low elastic recovery, and a high elongation at break.

The substrate 111 may include at least one of a polymeric film, a polymeric foam, a polymeric hydrocolloid, and a polymeric alginate. The substrate 111 may include polyolefins, such as polyethylene, including high density polyethylene, low density polyethylene, linear low density polyethylene, and linear ultra-low density polyethylene, polypropylene, and polybutylenes; vinyl copolymers, such as polyvinyl chlorides, both plasticized and unplasticized, and polyvinyl acetates; olefinic copolymers, such as ethylene/methacrylate copolymers, ethylene/vinyl acetate copolymers, acrylonitrile-butadiene-styrene copolymers, and ethylene propylene copolymers; acrylic polymers and copolymer; and combinations thereof. The substrate 111 may be in the form of single or multi-layer films, non-woven films, porous films, foam-like films, and combinations thereof. The substrate 111 may be fabricated using any suitable method of film forming, such as extrusion, co-extrusion, solvent casting, foaming, non-woven technology, and the like.

As shown in FIG. 3, the substrate 111 may include a first substrate portion 111A and a second substrate portion 111B. As shown in FIGS. 2 and 3, in some embodiments, the second substrate portion 111B may be disconnected from the first substrate portion 111A. Alternatively, the first substrate portion 111A and the second substrate portion 111B may be connected to each other, such that the substrate 111 is a single integral component.

The base 110 may further include at least one pull tab 116. In some embodiments, the at least one pull tab 116 may be disposed on the first major surface 112 of the substrate 111. Alternatively, the at least one pull tab 116 may extend from the substrate 111. The at least one pull tab 116 may be manually grippable. That is, the at least one pull tab 116 may be manually gripped by a medical practitioner to detach the base 110 from the skin 20.

In some embodiments, the at least one pull tab 116 may include a pair of pull tabs 116 disposed on the substrate 111 opposite to each other. The pair of pull tabs 116 may facilitate removal of the base 110 from the skin 20. Specifically, the pair of pull tabs 116 may be pulled in opposite directions to detach the base 110 from the skin 20.

As shown in FIG. 2, in some embodiments, the base 110 may include a pair of pull tabs 116 disposed on each of the first substrate portion 111A and the second substrate portion 111B that are disconnected from each other. Specifically, in some embodiments, the base 110 may include a first pair of pull tabs 116 at least partially disposed on the first major surface 112 of the first substrate portion 111A at opposite ends thereof, and a second pair of pull tabs 116 at least partially disposed on the first major surface 112 of the second substrate portion 111B at opposite ends thereof. In such embodiments, the pair of pull tabs 116 may facilitate removal of the first substrate portion 111A and the second substrate portion 111B from the skin 20 independently from each other.

The securement device 100 further includes a base mount 120 fixedly attached to the base 110. The base mount 120 may be fixedly attached to the first major surface 112 of the substrate 111. In other words, the base mount 120 may not move relative to the base 110. As a result, the base mount 120 may remain stationary relative to the skin 20 when the base 110 is detachably secured to the skin 20.

The base mount 120 includes a coupling member 122. The coupling member 122 may include a coupling base 122A, a mounting structure 122B extending from the coupling base 122A, and a coupling feature 122C disposed on the mounting structure 122B. The coupling base 122A shown in FIGS. 2 and 3 has a generally oval shape. However, it may be noted that the coupling base 122A may have any suitable shape, such as circular, rectangular, polygonal, and the like. In the illustrated embodiment of FIGS. 2 and 3, the coupling feature 122C includes a projection extending from the mounting structure 122B. In some other embodiments, the coupling feature 122C may include an aperture disposed on the mounting structure 122B.

The base mount 120 further includes a retaining member 124 spaced apart from the coupling member 122. The retaining member 124 may include a retaining base 124A and a wall 124B extending from the retaining base 124A. In some embodiments, the wall 124B may be perpendicular to the retaining base 124A. The wall 124B may define a cavity 124C (shown in FIG. 3). In some embodiments, the wall 124B may be substantially continuous. The retaining base 124A shown in FIGS. 2 and 3 has a generally oval shape. However, it may be noted that the retaining base 124A may include any suitable shape, such as, circular, rectangular, polygonal, and the like.

The first major surface 112 of the substrate 111 may be fixedly attached to the coupling member 122 and the retaining member 124. In some embodiments, the first substrate portion 111A may be fixedly attached to the coupling member 122. In some embodiments, the second substrate portion 111B may be fixedly attached to the retaining member 124.

The base mount 120 may further include a connecting member 123 (best shown in FIG. 3) that connects the coupling member 122 and the retaining member 124. In some embodiments, the base mount 120 may be a single integral molded part. In other words, in some embodiments, the coupling member 122, the connecting member 123, and the retaining member 124 may be integral with each other.

The securement device 100 further includes a swing arm 130. The swing arm 130 includes an elongate member 132 pivotally coupled to the coupling member 122 of the base mount 120. The elongate member 132 may be pivotally coupled to the coupling member 122 via the coupling feature 122C. Specifically, the elongate member 132 may be pivotally coupled to the mounting structure 122B via the coupling feature 122C. The elongate member 132 may include a complementary coupling feature corresponding to the coupling feature 122C of the base mount 120, such that the elongate member 132 can be pivotally coupled to the coupling member 122. For example, the elongate member 132 may include an aperture that receives and couples with the coupling feature 122C.

The elongate member 132 may include a first end 133A and a second end 133B opposite to the first end 133A. The elongate member 132 may extend between the first and second ends 133A, 133B thereof. As shown in FIG. 2, the elongate member 132 may be pivotally coupled to the coupling member 122 proximal to the first end 133A.

The swing arm 130 may be pivotable about a pivot axis 126 (also shown in FIG. 3). The pivot axis 126 may extend through the coupling feature 122C of the coupling member 122. Upon detachable securement of the base 110 to the skin 20 of the patient 22, the pivot axis 126 may intersect the insertion site 18. Upon detachable securement of the base 110 to the skin 20 of the patient 22, the pivot axis 126 may further intersect the cannula 16 at the insertion site 18. As shown in FIG. 2, the base mount 120 may further include one or more indicia 125 disposed on the coupling member 122. The one or more indicia 125 may be, for example, printed on the coupling member 122. Upon detachable securement of the base 110 to the skin 20 of the patient 22, the one or more indicia 125 may be aligned with the insertion site 18. Specifically, the base 110 may detachably secured to the skin 20, such that the one or more indicia 125 align with the insertion site 18. As shown in FIG. 2, the one or more indicia 125 may include a line disposed on the coupling base 122A of the coupling member 122. The line may be aligned with the pivot axis 126.

The elongate member 132 may further include a tapered portion 132A extending from the first end 133A and a curved portion 132B disposed adjacent to the tapered portion 132A. The tapered portion 132A may taper from the curved portion 132B towards the first end 133A. Furthermore, the curved portion 132B may curve away from the cannula securement member 134. This geometry of the swing arm 130 may allow the swing arm 130 to freely pivot relative to the base mount 120.

The swing arm 130 further includes a cannula securement member 134 connected to the elongate member 132 and spaced apart from the coupling member 122. As shown in FIG. 2, the cannula securement member 134 may be connected to the elongate member 132 proximal to the second end 133B of the elongate member 132. In some embodiments, the cannula securement member 134 may be connected to the elongate member 132 at the second end 133B of the elongate member 132. The cannula securement member 134 is configured to removably secure the cannula 16 thereto. The cannula securement member 134 is further configured to be detachably coupled to the retaining member 124. The geometry of the swing arm 130 described above may allow the swing arm 130 to freely pivot relative to the base mount 120 while the cannula 16 is detachably coupled to the cannula securement member 134 without obstruction.

The cannula securement member 134 may include a cannula receiving portion 136 connected to the elongate member 132. In some embodiments, the cannula receiving portion 136 may be connected to the elongate member 132 at the second end 133B of the elongate member 132.

As noted above, the cannula securement member 134 is configured to be detachably coupled to the retaining member 124. Upon detachable coupling of the cannula securement member 134 to the retaining member 124, the cannula receiving portion 136 of the cannula securement member 134 may be at least partially received and detachably secured within the cavity 124C of the retaining member 124. The wall 124B of the retaining member 124 may engage with the cannula receiving portion 136 to detachably couple the cannula securement member 134 to the retaining member 124. In some embodiments, the cannula receiving portion 136 of the swing arm 130 may detachably couple to the retaining member 124 by a snap-fit connection.

The cannula securement member 134 may further include a release member 131 extending from the cannula receiving portion 136. The release member 131 may be configured to be manually gripped for releasing the cannula receiving portion 136 from the retaining member 124. The release member 131 may have any suitable shape for gripping. The release member 131 may be gripped by a medical practitioner to detach the cannula receiving portion 136 from the retaining member 124. Upon detachment of the cannula securement member 134 from the retaining member 124, the swing arm 130 is pivotable relative to the base mount 120.

The cannula securement member 134 may further include a cap 138 configured to be detachably coupled to the cannula receiving portion 136. In FIG. 2, the cap 138 is in a coupled state 138A. In the coupled state 138A, the cap 138 may be detachably coupled to the cannula receiving portion 136.

In some embodiments, the cap 138 may be configured to be detachably coupled to the cannula receiving portion 136 by snap-fit connection. The cap 138 may include a release member 140 extending outwardly therefrom. The release member 140 may be manually gripped to detach the cap 138 from the cannula receiving portion 136. In some embodiments, the cap 138 may be pivotally coupled to the cannula receiving portion 136 or tethered to the cannula receiving portion 136. Alternatively, the cap 138 may be disconnected from the cannula receiving portion 136.

The cannula receiving portion 136 may include a first slot 137 (shown in FIG. 3) configured to at least partially receive the cannula 16 therein. Furthermore, the cap 138 may include a second slot 139. Upon detachable coupling of the cap 138 to the cannula receiving portion 136, the second slot 139 may be at least partially aligned with the first slot 137. Thus, upon insertion of the cannula 16 in the first slot 137 and detachable coupling of the cap 138 to the cannula receiving portion 136, the first and second slots 137, 139 may collectively and at least partially receive the cannula 16 therebetween, such that the cannula 16 is removably secured to the cannula securement member 134.

In some embodiments, the cannula securement member 134 may further include a first sleeve 135A at least partially received within the first slot 137 and a second sleeve 135B at least partially received within the second slot 139. Each of the first and second sleeves 135A, 135B may be made from a high friction material, such as rubber, polymers such as low-density polyethene (LDPE), high-density polyethene (HDPE), polypropylene (PP), and the like.

Upon insertion of the cannula 16 in the first slot 137 and detachable coupling of the cap 138 to the cannula receiving portion 136, each of the first sleeve 135A and the second sleeve 135B may frictionally engage with the cannula 16, such that the cannula 16 is removably secured to the cannula securement member 134. Frictional engagement of the first sleeve 135A and the second sleeve 135B with the cannula 16 may prevent and/or reduce a movement of the cannula 16 relative to the cannula securement member 134 and ensure that the catheter 10 remains in place.

In some embodiments, the swing arm 130 may be a single integral molded part. In other words, in some embodiments, the elongate member 132 and the cannula securement member 134 may be integral with each other.

As shown in FIG. 2, in the secured state 101 of the securement device 100, the cannula 16 is removably secured to the cannula securement member 134 and the cannula securement member 134 is detachably coupled to the retaining member 124.

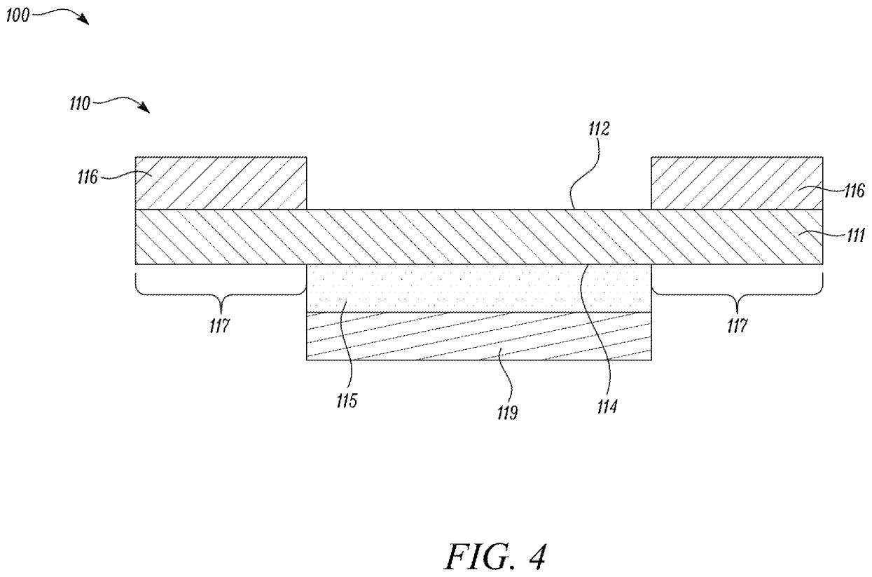

FIG. 4 illustrates a schematic sectional view of the securement device 100 taken along a line 1-1 of FIG. 2.

In some embodiments, the base 110 may further include an adhesive layer 115 at least partially disposed on the second major surface 114. The adhesive layer 115 may be configured to detachably secure the substrate 111 to the skin 20 of the patient 22. The adhesive layer 115 may be substantially continuous or patterned.

In some embodiments, the adhesive layer 115 may be at least partially disposed on the second major surface 114 of each of the first substrate portion 111A (shown in FIGS. 2 and 3) and the second substrate portion 111B (shown in FIGS. 2 and 3).

The second major surface 114 corresponding to the at least one pull tab 116 may be adhesive free and define an adhesive free zone 117. As shown in FIG. 4, in some embodiments, the second major surface 114 may define a pair of adhesive free zones 117 corresponding to the pair of pull tabs 116.

The adhesive layer 115 may include any suitable adhesive, such as a pressure sensitive adhesive (PSA). The adhesive layer 115 may include, for example, tackified rubber adhesives, such as natural rubber, olefins, silicones, polyisoprene, polybutadiene, polyurethanes, styrene-isoprene-styrene and styrene-butadiene-styrene block copolymers, and other elastomer; and tackified or untackified acrylic adhesives such as copolymers of iso-octyl acrylate and acrylic acid, which may be polymerized by radiation, solution, suspension, or emulsion techniques.

Preferably, the adhesive layer 115 may include a stretch-release adhesive. In some cases, an adhesive strength of the stretch-release adhesive may be less than a cohesive strength of the stretch-release adhesive, such that upon stretching of the substrate 111, adhesion capabilities of the stretch-release adhesive may essentially disappear. A tack of the stretch-release adhesive may be lost upon stretching of the substrate 111. Therefore, in some embodiments, the base 110 may be easily removed from the skin 20 of the patient 22 by gripping the at least one pull tab 116 to stretch the substrate 111.

The securement device 100 may further include a release liner 119 disposed on the adhesive layer 115. The release liner 119 may protect the adhesive layer 115 from contaminants, such as dust, debris, and the like, prior to use of the securement device 100. The release liner 119 may be peeled off from the adhesive layer 115 prior to removable securement of the base 110 to the skin 20 of the patient 22 (shown in FIG. 2). The release liner 119 may include any suitable material, such as Polyethylene Terephthalate (PET), High-Density Polyethylene (HDPE), Polyvinyl Chloride (PVC), Polypropylene (PP), and the like.

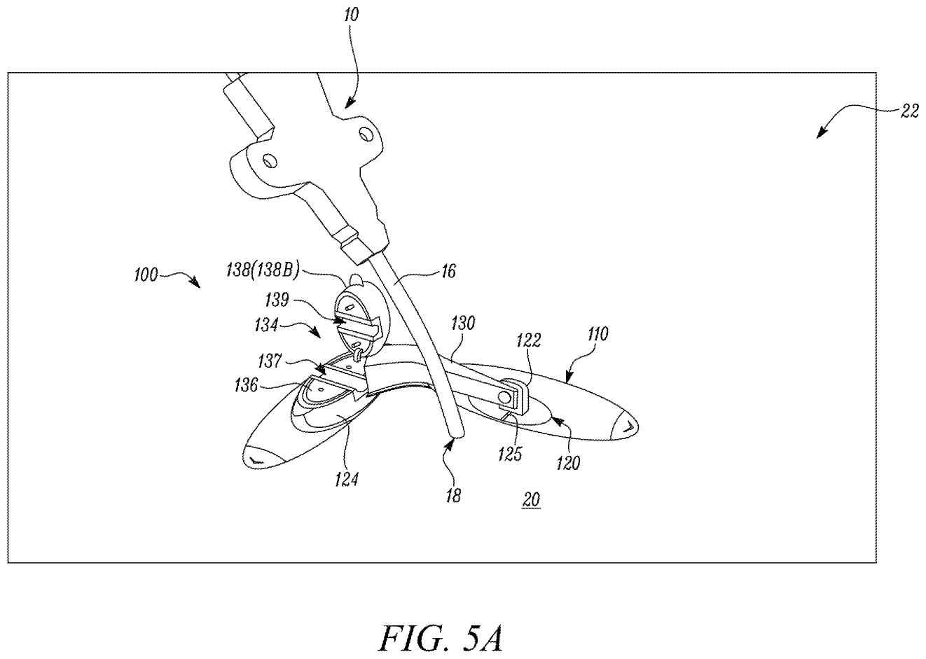

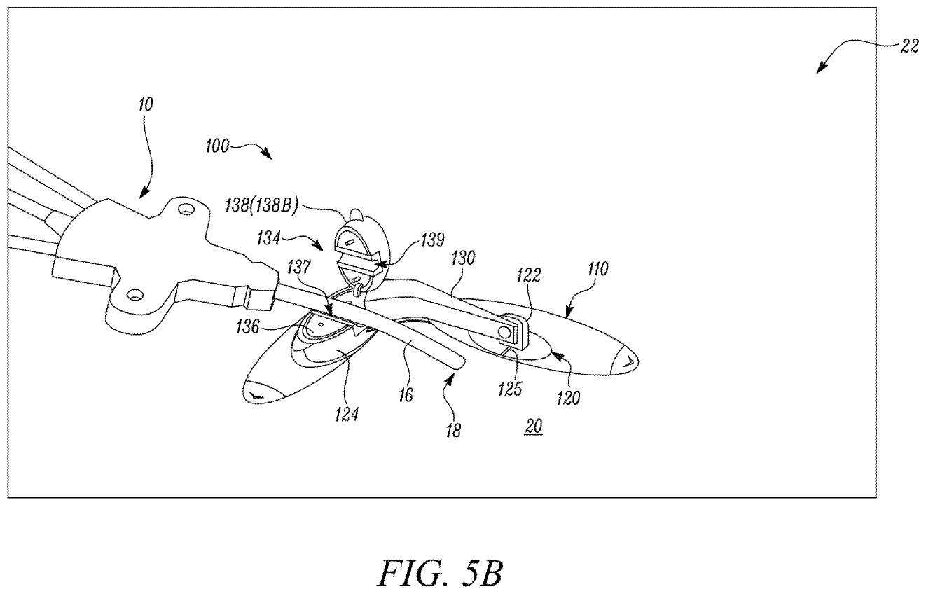

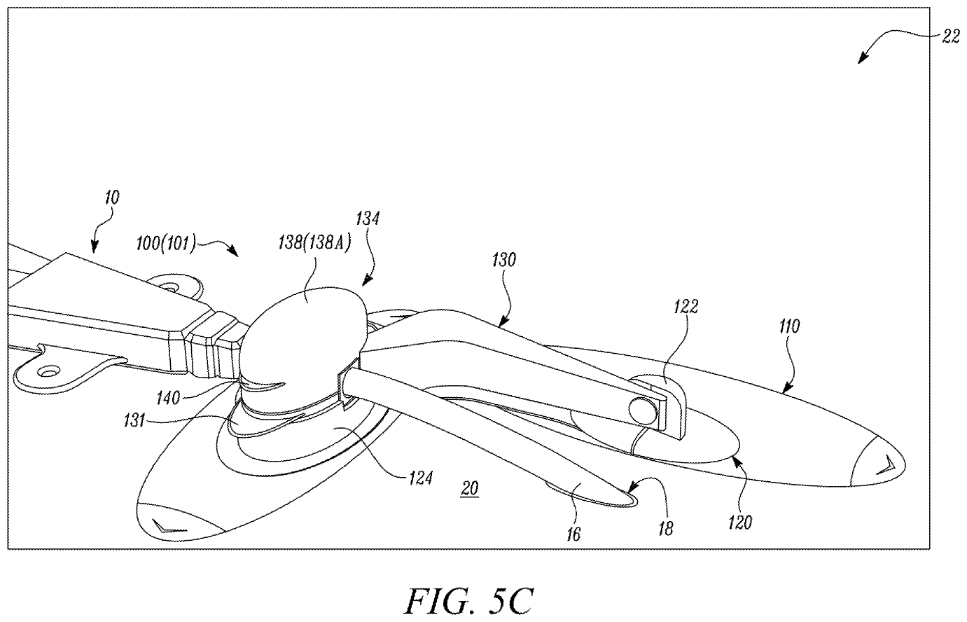

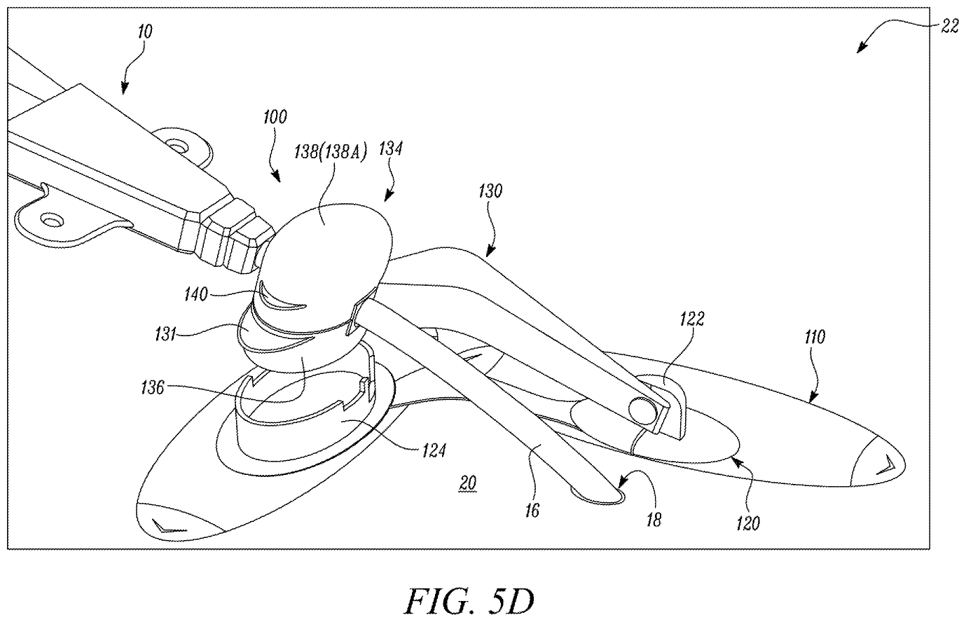

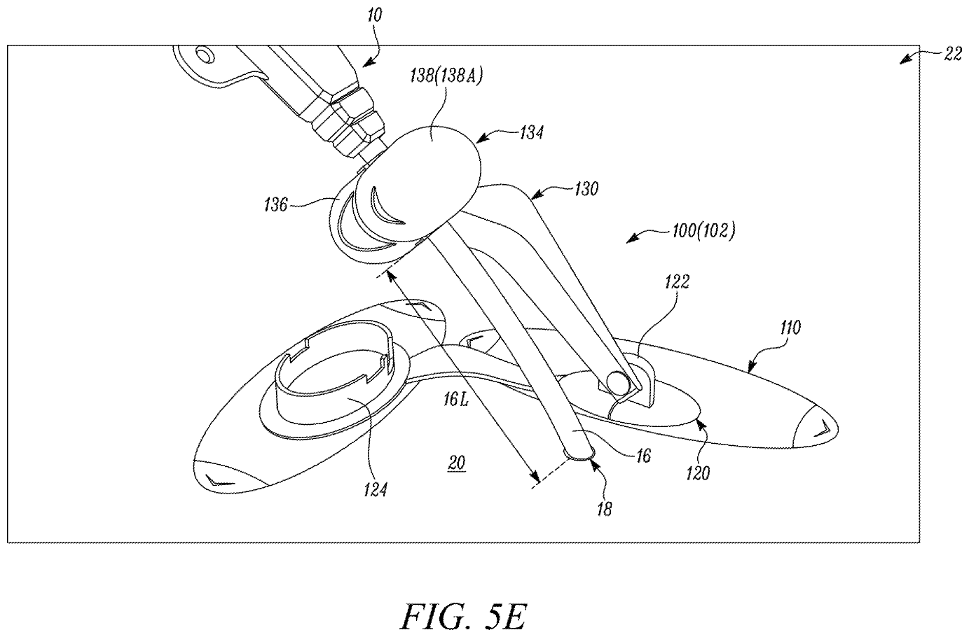

FIGS. 5A-5E illustrate schematic perspective views depicting a method of using the securement device 100 according to an embodiment of the present disclosure.

Referring to FIG. 5A, the cannula 16 of the catheter 10 may be inserted into the skin 20 of the patient 22 at the insertion site 18. Furthermore, the cannula receiving portion 136 of the swing arm 130 may be detachably coupled to the retaining member 124 of the base mount 120.

The base 110 may be removably secured to the skin 20 of the patient 22. The base 110 may be removably secured to the skin 20 of the patient 22 via the adhesive layer 115 (shown in FIG. 4). The base 110 may be removably secured to the skin 20 in such a way that the pivot axis 126 (shown in FIG. 2) intersects the insertion site 18. Removably securing the base 110 to the skin 20 in such a way that the pivot axis 126 intersects the insertion site 18 may allow the swing arm 130 to be pivoted relative to the base mount 120 without causing a pistoning movement of the cannula 16.

In some embodiments, the base 110 may be removably secured to the skin 20 in such a way that the one or more indicia 125 of the base mount 120 may be aligned with the insertion site 18. Moreover, the cap 138 may be in a detached state 138B. In the detached state 138B, the cap 138 may be detached from the cannula receiving portion 136. The cap 138 may be detached from the cannula receiving portion 136 by gripping and moving the release member 140.

Referring now to FIG. 5B, the cannula 16 of the catheter 10 may be at least partially received in the first slot 137 of the cannula receiving portion 136. Further, as shown in FIG. 5C, the cap 138 may be moved to the coupled state 138A from the detached state 138B. In the configuration shown in FIG. 5C, the securement device 100 is in the secured state 101. As discussed above, in the secured state 101, the cannula 16 is removably secured to the cannula securement member 134 and the cannula securement member 134 is detachably coupled to the retaining member 124.

In the secured state 101, the securement device 100 may firmly secure the catheter 10 to the skin 20 of the patient 22. The securement device 100 may therefore prevent various complications such as phlebitis, extravasation/infiltration, leakage, occlusion, and bloodstream infections that may occur due to movement of the catheter 10 relative to the skin 20.

Referring to FIG. 5D, the cannula securement member 134 may be detached from the retaining member 124. The cannula securement member 134 may be detached from the retaining member 124 by gripping and pulling the release member 131. As shown in FIG. 5D, after detaching the cannula securement member 134 from the retaining member 124, the swing arm 130 may be pivoted away from the retaining member 124. Specifically, after detaching the cannula securement member 134 from the retaining member 124, the swing arm 130 may be pivoted away from the retaining member 124 while the cannula 16 is secured to the cannula securement member 134.

Upon detachment of the cannula securement member 134 from the retaining member 124, the swing arm 130 may be pivoted relative to the base mount 120 so as to bring the securement device 100 to a maintenance state 102, as shown in FIG. 5E. Referring to FIG. 5E, in the maintenance state 102 of the securement device 100, the cannula securement member 134 is detached from the retaining member 124 and the swing arm 130 is pivoted away from the retaining member 124, such that the cannula securement member 134 is spaced apart from the retaining member 124 while being secured to the cannula 16.

In some embodiments, an exposed length 16L of the cannula 16 from the cannula securement member 134 to the insertion site 18 is substantially constant during pivoting of the swing arm 130 relative to the retaining member 124. The exposed length 16L may remain substantially constant during pivoting of the swing arm 130 relative to the retaining member 124 when the base 110 is removably secured to the skin 20 in such a way that the pivot axis 126 (shown in FIG. 2) intersects the insertion site 18. As a result, the securement device may transition between the secured state 101 and the maintenance state 102 without causing a pistoning movement of the cannula 16.

In the maintenance state 102, the securement device 100 may provide the medical practitioner with access to the insertion site 18. The medical practitioner may perform a maintenance procedure at the insertion site 18 in the maintenance state 102 of the securement device 100. The securement device 100 may allow the medical practitioner to carry out the maintenance procedure without needing to remove or detach the securement device 100 from the skin 20.

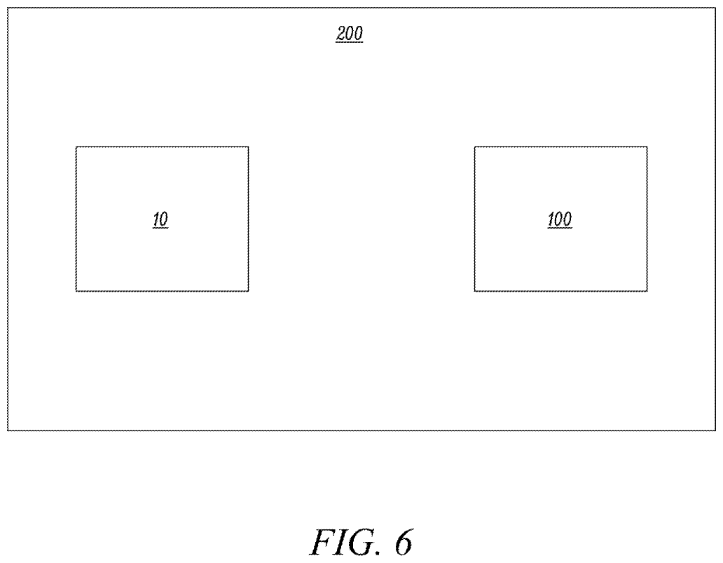

FIG. 6 illustrates schematic block diagram of a kit 200 according to an embodiment of the present disclosure.

The kit 200 includes the catheter 10 and the securement device 100. In some embodiments, the kit 200 may further include gloves. In some embodiments, the kit 200 may further include a sterilizing material. In some embodiments, the kit 200 may further include a cloth or other absorbent material. In some embodiments, the kit 200 may further an anti-microbial agent. The anti-microbial agent may be in the form of a liquid or a gel. In some embodiments, the kit 200 may further include cleaning articles, such as cleaning cloth, cotton balls, cotton swabs, and the like.

The kit 200 may be available to a medical practitioner for use in a sterilized package. The medical practitioner may remove the release liner 119 (shown in FIG. 4) and detachably couple the securement device 100 onto the skin 20. Referring to FIGS. 5A-5E and 6, the medical practitioner may detachably secure the base 110 of the securement device 100 to the skin 20, removably secure the cannula 16 to the cannula securement member 134, and detachably couple the cannula securement member 134 to the retaining member 124 to bring the securement device 100 to the secured state 101. The catheter 10 may thus be secured and stabilized. When required, the medical practitioner may detach the cannula securement member 134 from the retaining member 124 and pivot the swing arm 130 away from the retaining member 124 to bring the securement device 100 to the maintenance state 102. In the maintenance state 102, the insertion site 18 may be accessible to the medical practitioner. In the maintenance state 102, the securement device 100 may enable the medical practitioner to perform a maintenance procedure at the insertion site 18 without removal of the securement device 100 from the skin 20. The medical practitioner may subsequently perform the maintenance procedure.

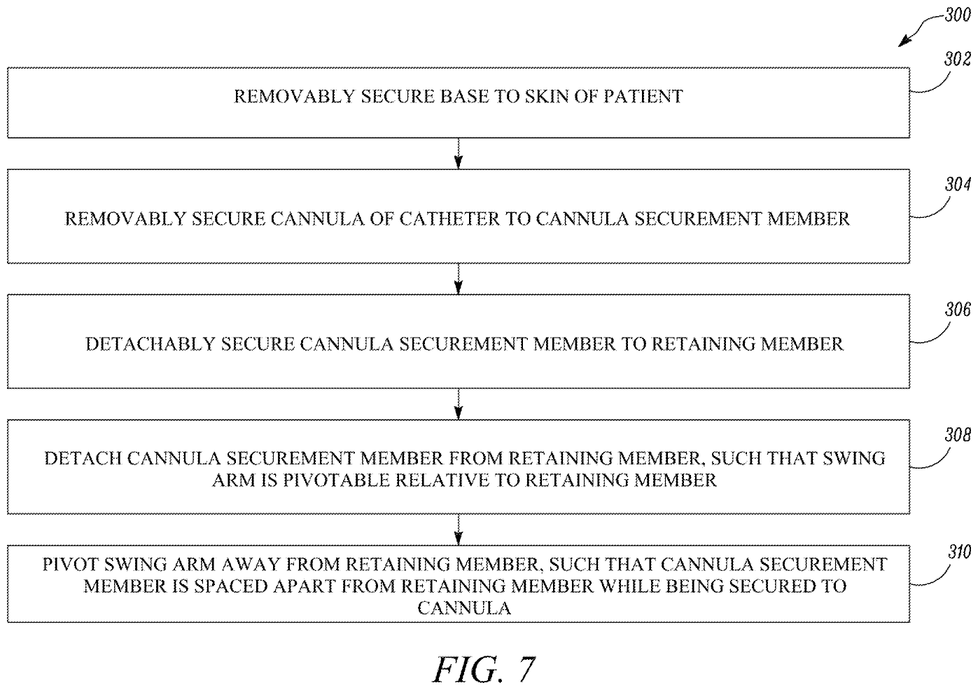

FIG. 7 illustrates a flowchart depicting various steps of a method 300 of using a securement device (e.g., the securement device 100 of FIG. 2) with a catheter (e.g., the catheter 10 shown in FIG. 1) having a cannula inserted into a skin of a patient at an insertion site according to an embodiment of the present disclosure. The securement device includes a base configured to be detachably secured to the skin of the patient. The securement device further includes a base mount fixedly attached to the base and having a coupling member and a retaining member spaced apart from the coupling member. The securement device further includes a swing arm having an elongate member pivotally coupled to the coupling member of the base mount and a cannula securement member connected to the elongate member and spaced apart from the coupling member. The method 300 will be further described with further reference to FIGS. 5A-5E.

At step 302, the method 300 includes removably securing the base to the skin of the patient. For example, as shown in FIG. 5A, the method 300 may include removably securing the base 110 to the skin 20 of the patient 22.

In some embodiments, removably securing the base to the skin of the patient comprises removably bonding the base to the skin of the patient via an adhesive layer. For example, in some embodiments, removably securing the base 110 to the skin 20 of the patient 22 may include removably bonding the base 110 to the skin 20 of the patient 22 via the adhesive layer 115 (shown in FIG. 4).

In some embodiments, removably securing the base to the skin of the patient includes aligning a pivot axis of the swing arm with the insertion site, such that the pivot axis intersects the insertion site. For example, as shown in FIGS. 2 and 5A, removably securing the base 110 to the skin 20 of the patient 22 may include aligning the pivot axis 126 of the swing arm 130 with the insertion site 18, such that the pivot axis 126 intersects the insertion site 18. Aligning the pivot axis 126 of the swing arm 130 with the insertion site 18, such that the pivot axis 126 intersects the insertion site 18 may allow the swing arm 130 to be pivoted relative to the base mount 120 without causing a pistoning movement of the cannula 16.

At step 304, the method 300 further includes removably securing the cannula of the catheter to the cannula securement member. For example, as shown in FIG. 5C, the method 300 may include removably securing the cannula 16 of the catheter 10 to the cannula securement member 134.

In some embodiments, removably securing the cannula of the catheter to the cannula securement member may include inserting the cannula at least partially in a first slot of a cannula receiving portion of the cannula securement member, and removably coupling a cap to the cannula receiving portion, such that the first slot of the cannula receiving portion and a second slot of the cap collectively and at least partially receive the cannula therebetween.

For example, as shown in FIG. 5B, removably securing the cannula 16 of the catheter 10 to the cannula securement member 134 may include inserting the cannula 16 at least partially in the first slot 137 of the cannula receiving portion 136 of the cannula securement member 134, and removably coupling the cap 138 to the cannula receiving portion 136, such that the first slot 137 of the cannula receiving portion 136 and the second slot 139 of the cap 138 collectively and at least partially receive the cannula 16 therebetween.

At step 306, the method 300 further includes detachably securing the cannula securement member to the retaining member. For example, as shown in FIG. 5C, the method 300 may include detachably securing the cannula securement member 134 to the retaining member 124. In some embodiments, the cannula securement member 134 may be detachably secured to the retaining member 124 while steps 302 and 304 are performed.

At step 308, the method 300 further includes detaching the cannula securement member from the retaining member, such that the swing arm is pivotable relative to the retaining member. For example, as shown in FIG. 5D, the method 300 may include detaching the cannula securement member 134 from the retaining member 124, such that the swing arm 130 is pivotable relative to the retaining member 124.

At step 310, the method 300 further includes pivoting the swing arm away from the retaining member, such that the cannula securement member is spaced apart from the retaining member while being secured to the cannula. For example, as shown in FIG. 5E, the method 300 may include pivoting the swing arm 130 away from the retaining member 124, such that the cannula securement member 134 is spaced apart from the retaining member 124 while being secured to the cannula 16.

In some embodiments, the method 300 further includes performing a maintenance procedure at the insertion site after pivoting the swing arm away from the retaining member. In some embodiments, the method 300 further includes pivoting the swing arm towards the retaining member after performing the maintenance procedure. In some embodiments, the method 300 further includes detachably coupling the cannula securement member to the retaining member.

For example, the method 300 may include performing the maintenance procedure at the insertion site 18 after pivoting the swing arm 130 away from the retaining member 124.

The method 300 may further include pivoting the swing arm 130 towards the retaining member 124 after performing the maintenance procedure. The method 300 may further include detachably coupling the cannula securement member 134 to the retaining member 124.

The securement device 100 of the present disclosure may firmly secure the catheter 10 to the skin 20 of the patient 22 in the secured state 101. Thus, the securement device 100 may prevent dislodgement and accidental removal of the catheter 10 from the skin 20 of the patient 22. Further, the securement device 100 may prevent various complications such as phlebitis, extravasation/infiltration, leakage, occlusion, and bloodstream infections that may otherwise occur due to movement of the catheter 10. In the maintenance state 102, the securement device 100 may enable a medical practitioner to perform a maintenance procedure at the insertion site 18 without removal of the securement device 100 from the skin 20. The securement device 100 may transition between the secured state 101 and the maintenance state 102 without causing a pistoning movement of the cannula 16. Thus, the securement device 100 may facilitate performing a maintenance procedure.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A securement device for a catheter having a cannula configured to be inserted into a skin of a patient at an insertion site, the securement device comprising:
a base configured to be detachably secured to the skin of the patient;
a base mount fixedly attached to the base, the base mount comprising a coupling member and a retaining member spaced apart from the coupling member; and
a swing arm comprising:
an elongate member pivotally coupled to the coupling member of the base mount; and
a cannula securement member connected to the elongate member and spaced apart from the coupling member, wherein the cannula securement member is configured to removably secure the cannula thereto, and wherein the cannula securement member is further configured to be detachably coupled to the retaining member;
wherein, upon detachment of the cannula securement member from the retaining member, the swing arm is pivotable relative to the base mount;
wherein, in a secured state of the securement device, the cannula is removably secured to the cannula securement member and the cannula securement member is detachably coupled to the retaining member; and
wherein, in a maintenance state of the securement device, the cannula securement member is detached from the retaining member and the swing arm is pivoted away from the retaining member, such that the cannula securement member is spaced apart from the retaining member while being secured to the cannula.

2. The securement device of claim 1, wherein the base comprises:
a substrate comprising a first major surface fixedly attached to the coupling member and the retaining member, and a second major surface opposite to the first major surface;
an adhesive layer at least partially disposed on the second major surface and configured to detachably secure the substrate to the skin of the patient.

3. The securement device of claim 2, wherein the adhesive layer comprises a stretch-release adhesive.

4. The securement device of claim 2, wherein the substrate comprises:
a first substrate portion fixedly attached to the coupling member; and
a second substrate portion fixedly attached to the retaining member;
wherein the second substrate portion is disconnected from the first substrate portion, and wherein the adhesive layer is at least partially disposed on the second major surface of each of the first substrate portion and the second substrate portion.

5. The securement device of claim 1, wherein the base mount further comprises a connecting member that connects the coupling member and the retaining member.

6. The securement device of claim 1, wherein the cannula securement member comprises:
a cannula receiving portion connected to the elongate member and comprising a first slot configured to at least partially receive the cannula therein; and
a cap configured to be detachably coupled to the cannula receiving portion and comprising a second slot, wherein, upon detachable coupling of the cap to the cannula receiving portion, the second slot is at least partially aligned with the first slot.

7. The securement device of claim 6, wherein, upon insertion of the cannula in the first slot and detachable coupling of the cap to the cannula receiving portion, the first and second slots collectively and at least partially receive the cannula therebetween, such that the cannula is removably secured to the cannula securement member.

8. The securement device of claim 6, wherein the cannula securement member further comprises a first sleeve at least partially received within the first slot and a second sleeve at least partially received within the second slot, wherein, upon insertion of the cannula in the first slot and detachable coupling of the cap to the cannula receiving portion, each of the first sleeve and the second sleeve frictionally engages with the cannula, such that the cannula is removably secured to the cannula securement member.

9. The securement device of claim 6, wherein the cap is pivotally coupled to the cannula receiving portion or tethered to the cannula receiving portion.

10. The securement device of claim 6, wherein the cannula receiving portion of the swing arm detachably couples to the retaining member by a snap-fit connection.

11. The securement device of claim 1, wherein the cannula securement member further comprises a release member extending from the cannula receiving portion, and wherein the release member is configured to be manually gripped for releasing the cannula receiving portion from the retaining member.

12. The securement device of claim 1, wherein the elongate member comprises a first end and a second end opposite to the first end, wherein the elongate member is pivotally coupled to the coupling member proximal to the first end, and wherein the cannula securement member is connected to the elongate member proximal to the second end.

13. The securement device of claim 12, wherein the elongate member further comprises a tapered portion extending from the first end and a curved portion disposed adjacent to the tapered portion, wherein the tapered portion tapers from the curved portion towards the first end, and wherein the curved portion curves away from the cannula securement member.

14. The securement device of claim 1, wherein the swing arm is a single integral molded part.

15. The securement device of claim 1, wherein the base mount further comprises one or more indicia disposed on the coupling member, and wherein, upon detachable securement of the base to the skin of the patient, the one or more indicia are aligned with the insertion site.

16. The securement device of claim 1, wherein the swing arm is pivotable about a pivot axis, and wherein, upon detachable securement of the base to the skin of the patient, the pivot axis intersects the insertion site.

17. A kit comprising:
a catheter comprising a cannula configured to be inserted into a skin of a patient at an insertion site; and
a securement device comprising:
a base configured to be detachably secured to the skin of the patient;
a base mount fixedly attached to the base, the base mount comprising a coupling member and a retaining member spaced apart from the coupling member; and
a swing arm comprising:
an elongate member pivotally coupled to the coupling member of the base mount; and
a cannula securement member connected to the elongate member and spaced apart from the coupling member, wherein the cannula securement member is configured to removably secure the cannula thereto, and wherein the cannula securement member is further configured to be detachably coupled to the retaining member;
wherein, upon detachment of the cannula securement member from the retaining member, the swing arm is pivotable relative to the base mount;
wherein, in a secured state of the securement device, the cannula is removably secured to the cannula securement member and the cannula securement member is detachably coupled to the retaining member; and
wherein, in a maintenance state of the securement device, the cannula securement member is detached from the retaining member and the swing arm is pivoted away from the retaining member, such that the cannula securement member is spaced apart from the retaining member while being secured to the cannula.

18. The kit of claim 17, wherein the base comprises:
a substrate comprising a first major surface fixedly attached to the coupling member and the retaining member, and a second major surface opposite to the first major surface;
an adhesive layer at least partially disposed on the second major surface and configured to detachably secure the substrate to the skin of the patient.

19. The kit of claim 18, wherein the adhesive layer comprises a stretch-release adhesive.

20. The kit of claim 18, wherein the substrate comprises:
a first substrate portion fixedly attached to the coupling member; and
a second substrate portion fixedly attached to the retaining member;
wherein the second substrate portion is disconnected from the first substrate portion, and wherein the adhesive layer is at least partially disposed on the second major surface of each of the first substrate portion and the second substrate portion.

21-38. (canceled)
